(19) United States
(12) Patent Application Publication
(10) Pub. No.: US 2024/0346646 A1
(43) Pub. Date: Oct. 17, 2024

(54) SYSTEM AND METHOD FOR AUTOMATICALLY ADJUSTING A POSITION OF AN IMAGING SYSTEM

(71) Applicant: Medtronic Navigation, Inc., Louisville, CO (US)

(72) Inventors: Dany Junio, Brookline, MA (US); Erica C. Freund, Cambridge, MA (US); Elizabeth R. Rooney, Waltham, MA (US); Alain C. Pierre, Stoughton, MA (US)

(21) Appl. No.: 18/608,417

(22) Filed: Mar. 18, 2024

Related U.S. Application Data
(60) Provisional application No. 63/458,697, filed on Apr. 12, 2023.
(60) Provisional application No. 63/458,694, filed on Apr. 12, 2023.

(51) Int. Cl. G06T 7/00; A61B 6/08; A61B 6/10; G06T 7/11; G06T 7/55; G06T 7/73

(52) U.S. Cl. CPC G06T 7/0012; A61B 6/08; A61B 6/107; G06T 7/11; G06T 7/55; G06T 7/73; G06T 2207/10116; G06T 2207/30004

(57) ABSTRACT

A method and system for positioning an imaging system includes positioning the imaging system, acquiring a first image, detecting a first body structure in the first image, determining a first position of the imaging system relative to the first body structure based on the first image, determining a distance to a target image position based on the relative position, and moving the imaging system toward the target image position.

### CROSS-REFERENCE TO RELATED APPLICATIONS

This application claims the benefit of and priority to U.S. Provisional Patent Application No. 63/458,697 filed Apr. 12, 2023 and U.S. Provisional Patent Application No. 63/458,694 filed Apr. 12, 2023, and the disclosures of each of the above-identified applications are hereby incorporated by reference in their entirety.

### FIELD

The present disclosure relates to imaging a subject, and particularly to a system to automatically move the imager to a position relative to anatomy.

### BACKGROUND

This section provides background information related to the present disclosure which is not necessarily prior art.

A subject, such as a human patient, may undergo a procedure. The procedure may include a surgical procedure to correct or augment an anatomy of the subject. The augmentation of the anatomy can include various procedures, such as movement or augmentation of bone, insertion of an implant (i.e., an implantable device), or other appropriate procedures.

When using an imager, positioning the imager in a location to align the anatomical volume into the scan volume is important. Reducing the amount of time to position the imager and therefore reducing the amount of radiation to a patient is also important. Typically, a technician tries to manually position the imager by looking at a two-dimensional image. Using trial and adjustment, the technician adjusts the position of the imager until the desired image is obtained.

### SUMMARY

This section provides a general summary of the disclosure, and is not a comprehensive disclosure of its full scope or all of its features.

According to various embodiments, a system to acquire image data of a subject may be an imaging system that uses x-rays. The subject may be a living patient (e.g., a human patient). The subject may also be a non-living subject, such as an enclosure, a casing, etc. Generally, the imaging system may acquire image data of an interior of the subject. The imaging system may include a moveable source and/or detector that is moveable relative to the subject. The position and movement of the system is performed automatically to reduce the overall imaging time and provide less exposure of x-rays to the subject. In various embodiments, a method and system for positioning an imaging system includes positioning the imaging system, acquiring a first image, detecting a first body structure in the first image, determining a first position of the imaging system relative to the first body structure based on the first image, determining a distance to a target image position based on the relative position, and moving the imaging system toward the target image position.

In another aspect of the disclosure, a system to move an imaging system, the system has a controller configured to execute instructions to, acquire a first image, detect a first body structure in the first image, determine a first position of the imaging system relative to the first body structure based on the first image, determine a distance to a target image position based on the relative position and move the imaging system toward the target image position.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is an environmental view of an imaging system in an operating theatre;

FIG. 2 is a detailed schematic view of an imaging system with a source and detector configured to move around a subject, according to various embodiments;

FIG. 3 is a block diagrammatic view of the imaging system.

FIG. 4 is an exemplary illustration of a labeled and classified image;

FIG. 5 is a flowchart of a method for operating the system.

Corresponding reference numerals indicate corresponding parts throughout the several views of the drawings.

### DETAILED DESCRIPTION

Example embodiments will now be described more fully with reference to the accompanying drawings.

A subject may be imaged with an imaging system, as discussed further herein. The subject may be a living subject, such as a human patient. Image data may be acquired of the human patient and may be combined to provide an image of the human patient that is greater than any dimension of any single projection acquired with the imaging system. It is understood, however, that image data may be acquired of a non-living subject, such an inanimate subject including a housing, casing, interior of a super structure, or the like. For example, image data may be acquired of an airframe for various purposes, such as diagnosing issues and/or planning repair work.

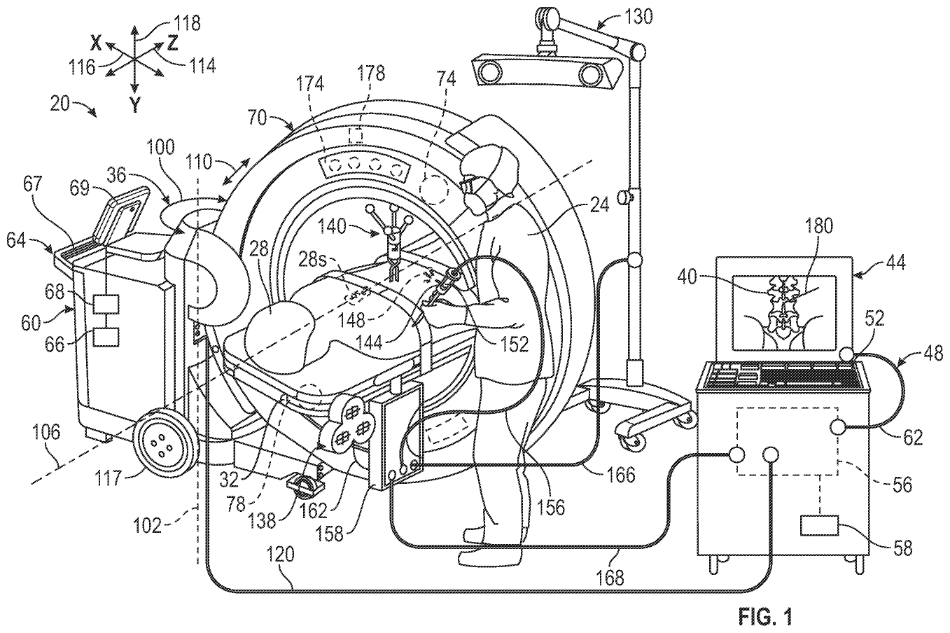

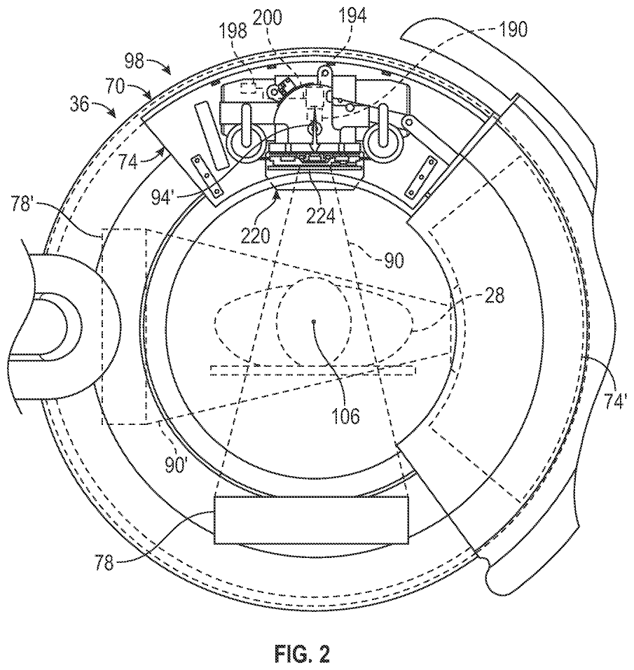

With reference to FIGS. 1 and 2, a schematic view of a procedure room 20 is illustrated. A user 24, such as a surgeon, can perform a procedure on a subject, such as a patient 28. The subject may be placed on a support, such as a table 32 for a selected portion of the procedure. The table 32 may not interfere with image data acquisition with an imaging system 36. In performing the procedure, the user 24 can use the imaging system 36 to acquire image data of the patient 28 to allow a selected system to generate or create images to assist in performing a procedure. Images generated with the image data may be two-dimensional (2D) images, three-dimensional (3D), or appropriate type of images, such as a model (such as a three-dimensional (3D) image), long views, single projections views, etc. can be generated using the image data and displayed as an image 40 on a display device 44. The display device 44 can be part of and/or connected to a processor system 48 that includes a user interface 52, such as a keyboard, mouse, stylus, a touch screen as part of the display device 44 or combinations thereof, and a processor 56, which can include one or more processors, processor module, and/or microprocessors incorporated with the processing system 48 along with selected types of non-transitory and/or transitory memory 58. A connection 62 can be provided between the processor 56 and the display device 44 for data communication to allow driving the display device 44 to display or illustrate the image 40. The processor 56 may be any appropriate type of processor such as a general-purpose processor that executes instructions included in a program or an application specific processor such as an application specific integrated circuit.

The imaging system 36 can include but is not limited to an O-Arm® imaging system sold by Medtronic Navigation, Inc. having a place of business in Louisville, CO, USA. The imaging system 36, including the O-Arm® imaging system, or other appropriate imaging systems may be in use during a selected procedure, such as the imaging system described in U.S. Patent App. Pubs. 2012/0250822, 2012/0099772, and 2010/0290690, all the above incorporated herein by reference.

The imaging system 36, when, for example, including the O-Arm® imaging system, may include a mobile cart 60 that includes a controller and/or control system 64. The control system 64 may include a processor and/or processor system 68 (similar to the processor 56), a user interface 67 such as a keyboard, a mouse, a touch screen, a memory 58 (e.g., a non-transitory memory) and a display device 69. The memory system 66 may include various instructions that are executed by the processor 68 that acts as a controller to control the imaging system 36, including various portions of the imaging system 36.

The imaging system 36 may include further additional portions, such as an imaging gantry 70 in which is positioned a source unit (also referred to as a source assembly) 74 and a detector unit (also referred to as a detector assembly) 78. In various embodiments, the detector 78 alone and/or together with the source unit 74 may be referred to as an imaging head of the imaging system 36. The gantry 70 is moveably connected to the mobile cart 60. The gantry 70 may be O-shaped or toroid shaped, wherein the gantry 70 is substantially annular and includes walls that form a volume in which the source unit 74 and detector 78 may move. Other imaging system may include gantry or imaging portions that are not O-shaped. The mobile cart 60 may also be moved. In various embodiments, the gantry 70 and/or the cart 60 may be moved while image data is acquired, including both being moved simultaneously. Also, the imaging system 36, via the mobile cart 60, can be moved from one operating theater to another (e.g., another room). The gantry 70 can move relative to the cart 60, as discussed further herein. This allows the imaging system 36 to be mobile and moveable relative to the subject 28, thus allowing it to be used in multiple locations and with multiple procedures without requiring a capital expenditure or space dedicated to a fixed imaging system.

The processor 68 may be a general-purpose processor or an application specific application processor. The memory system 66 may be a non-transitory memory such as a spinning disk or solid-state non-volatile memory. In various embodiments, the memory system may include instructions to be executed by the processor 68 to perform functions and determine results, as discussed herein. The memory system 66 may be used to store images from the imaging system 36 to allow calculations to be performed thereon. The memory system 66 may be used to store intermediate and final calculations, such as data for identifying body structures, distance for the imaging system to travel, a target position for the imaging system 36.

In various embodiments, the imaging system 36 may include an imaging system that acquires images and/or image data by the use of emitting x-rays and detecting x-rays after interactions and/or attenuations of the x-rays with or by the subject 28. The x-ray imaging may be an imaging modality. It is understood that other imaging modalities are possible, such as other high energy beams, etc.

Thus, in the imaging system 36, the source unit 74 may be an x-ray emitter that can emit x-rays at and/or through the patient 28 to be detected by the detector 78. As is understood by one skilled in the art, the x-rays emitted by the source 74 can be emitted in a cone 90 along a selected main vector 94 and detected by the detector 78, as illustrated in FIG. 2. The source 74 and the detector 78 may also be referred to together as a source/detector unit 98, especially wherein the source 74 is generally diametrically opposed (e.g., 180 degrees (°) apart) from the detector 78 within the gantry 70.

The imaging system 36 may move, as a whole or in part, relative to the subject 28. The imaging system 36, including portions or parts thereof, may move in appropriate manners as discussed herein. In various embodiments, the imaging system may move as disclosed in U.S. Pat. No. 8,768,029, incorporated herein by reference. For example, the source 74 and the detector 78 can move around the patient 28, e.g., a 360°motion, spiral, portion of a circle, etc. The movement of the source/detector unit 98 within the gantry 70 may allow the source 74 to remain generally 180°opposed (such as with a fixed inner gantry or rotor or moving system) to the detector 78. Thus, the detector 78 may be referred to as moving around (e.g., in a circle or spiral) the subject 28 and it is understood that the source 74 remains opposed thereto, unless disclosed otherwise.

Also, the gantry 70 can move isometrically (also referred as “wag”) relative to the subject 28 generally in the direction of arrow 100 around an axis 102, such as through the cart 60, as illustrated in FIG. 1. The gantry 70 can also tilt relative to a longitudinal axis 106 of the patient 28 illustrated by arrows 110. In tilting, a plane of the gantry 70 may tilt or form a non-orthogonal angle with the axis 106 of the subject 28.

The gantry 70 may also move longitudinally in the direction of arrows 114 along the line 106 relative to the subject 28 and/or the cart 60. Also, the cart 60 may move to move the gantry 70. Further, the gantry 70 can move up and down generally in the Y-axis direction of arrows 118 relative to the cart 60 and/or the subject 28, generally transverse to the axis 106 and parallel with the axis 102. The gantry may also be moved in an X direction in the direction of the arrows 116 by moving the wheels 117.

The movement of the imaging system 36, in whole or in part is to allow for positioning of the source/detector unit (SDU) 98 relative to the subject 28. The imaging system 36 can be precisely controlled to move the SDU 98 relative to the subject 28 to generate precise image data of the subject 28. The imaging system 36 can be connected to the processor 56 via a connection 120, which can include a wired or wireless connection or physical media transfer from the imaging system 36 to the processor 56. Thus, image data collected with the imaging system 36 can be transferred to the processing system 56 for navigation, display, reconstruction, etc.

The source 74, as discussed herein, may include one or more sources of x-rays for imaging the subject 28. In various embodiments, the source 74 may include a single source that may be powered by more than one power source to generate and/or emit x-rays at different energy characteristics. Further, more than one x-ray source may be the source 74 that may be powered to emit x-rays with differing energy characteristics at selected times.

According to various embodiments, the imaging system 36 can be used with an un-navigated or navigated procedure. In a navigated procedure, a localizer and/or digitizer, including either or both of an optical localizer 130 and/or an electromagnetic localizer 138 can be used to generate a field and/or receive and/or send a signal within a navigation domain relative to the subject 28. The navigated space or navigational domain relative to the subject 28 can be registered to the image 40. Correlation, as understood in the art, is to allow registration of a navigation space defined within the navigational domain and an image space defined by the image 40. A patient tracker or dynamic reference frame 140 can be connected to the subject 28 to allow for a dynamic registration and maintenance of registration of the subject 28 to the image 40. It is understood by one skilled in the art that the subject disclosure relates to registration according to various embodiments including tracking systems that either or both of optical or EM and registration may occur as discussed herein. Registration may also be automatic based on tracking the imaging system, such as the O-Arm® imaging system.

The patient tracking device or dynamic registration device 140 and an instrument 144 can then be tracked relative to the subject 28 to allow for a navigated procedure. The instrument 144 can include a tracking device, such as an optical tracking device 148 and/or an electromagnetic tracking device 152 to allow for tracking of the instrument 144 with either or both of the optical localizer 130 or the electromagnetic localizer 138. A navigation/probe interface device 158 may have communications (e.g., wired or wireless) with the instrument 144 (e.g., via a communication line 156), with the electromagnetic localizer 138 (e.g., via a communication line 162), and/or the optical localizer 130 (e.g., via a communication line 166). The interface 158 can also communicate with the processor 56 with a communication line 168 and may communicate information (e.g., signals) regarding the various items connected to the interface 158. It will be understood that any of the communication lines can be wired, wireless, physical media transmission or movement, or any other appropriate communication. Nevertheless, the appropriate communication systems can be provided with the respective localizers to allow for tracking of the instrument 144 relative to the subject 28 to allow for illustration of a tracked location of the instrument 144 relative to the image 40 for performing a procedure.

One skilled in the art will understand that the instrument 144 may be any appropriate instrument, such as a ventricular or vascular stent, spinal implant, neurological stent or stimulator, ablation device, or the like. The instrument 144 can be an interventional instrument or can include or be an implantable device. Tracking the instrument 144 allows for viewing a location (including x,y,z position and orientation) of the instrument 144 relative to the subject 28 with use of the registered image 40 without direct viewing of the instrument 144 within the subject 28.

Further, the imaging system 36, such as the gantry 70, can include an optical tracking device 174 and/or an electromagnetic tracking device 178 to be tracked with the respective optical localizer 130 and/or electromagnetic localizer 138. Accordingly, the imaging system 36 can be tracked relative to the subject 28 as can the instrument 144 to allow for initial registration, automatic registration, or continued registration of the subject 28 relative to the image 40. Registration and navigated procedures are discussed in the above incorporated U.S. Pat. No. 8,238,631, incorporated herein by reference. Upon registration and tracking of the instrument 144, an icon 180 may be displayed relative to, including overlaid on, the image 40. The image 40 may be an appropriate image and may include a 2D image, a 3D image, or any appropriate image as discussed herein.

With continuing reference to FIG. 2, according to various embodiments, the source 74 can include a single assembly that may include a single x-ray tube 190 that can be connected to a switch 194 that can interconnect a first power source 198 via a connection or power line 200. As discussed above, x-rays can be emitted from the x-ray tube 190 generally in the cone shape 90 towards the detector 78 and generally in the direction from the x-ray tube 190 as indicated by arrow, beam arrow, beam or vector 94. The switch 194 can switch power on or off to the tube 190 to emit x-rays of selected characteristics, as is understood by one skilled in the art. The vector 94 may be a central vector or ray within the cone 90 of x-rays. An x-ray beam may be emitted as the cone 90 or other appropriate geometry. The vector 94 may include a selected line or axis relevant for further interaction with the beam, such as with a filter member, as discussed further herein.

The subject 28 can be positioned within the x-ray cone 90 to allow for acquiring image data of the subject 28 based upon the emission of x-rays in the direction of vector 94 towards the detector 78. The x-ray tube 190 may be used to generate two-dimensional (2D) x-ray projections of the subject 28, including selected portions of the subject 28, or any area, region or volume of interest, in light of the x-rays impinging upon or being detected on a 2D or flat panel detector, as the detector 78. The 2D x-ray projections can be reconstructed, as discussed herein, to generate and/or display three-dimensional (3D) volumetric models of the subject 28, selected portion of the subject 28, or any area, region or volume of interest. As discussed herein, the 2D x-ray projections can be image data acquired with the imaging system 36, while the 3D volumetric models can be generated or model image data.

For reconstructing or forming the 3D volumetric image, appropriate techniques include Expectation maximization (EM), Ordered Subsets EM (OS-EM), Simultaneous Algebraic Reconstruction Technique (SART) and Total Variation Minimization (TVM), as generally understood by those skilled in the art. Various reconstruction techniques may also and alternatively include machine learning systems and algebraic techniques. The application to perform a 3D volumetric reconstruction based on the 2D projections allows for efficient and complete volumetric reconstruction. Generally, an algebraic technique can include an iterative process to perform a reconstruction of the subject 28 for display as the image 40. For example, a pure or theoretical image data projection, such as those based on or generated from an atlas or stylized model of a “theoretical” patient, can be iteratively changed until the theoretical projection images match the acquired 2D projection image data of the subject 28. Then, the stylized model can be appropriately altered as the 3D volumetric reconstruction model of the acquired 2D projection image data of the selected subject 28 and can be used in a surgical intervention, such as navigation, diagnosis, or planning. The theoretical model can be associated with theoretical image data to construct the theoretical model. In this way, the model or the image data 40 can be built based upon image data acquired of the subject 28 with the imaging system 36.

With continuing reference to FIG. 2, the source 74 may include various elements or features that may be moved relative to the x-ray tube 190. In various embodiments, for example, a collimator 220 may be positioned relative to the x-ray tube 190 to assist in forming the cone 90 relative to the subject 28. The collimator 220 may include various features such as movable members that may assist in positioning one or more filters within the cone 90 of the x-rays prior to reaching the subject 28. One or more movement systems 224 may be provided to move all and/or various portions of the collimator 220. Further, as discussed further herein, various filters may be used to shape the x-ray beam, such as shaping the cone 90, into a selected shape prior to reaching the subject 28. In various embodiments, as discussed herein, the x-rays may be formed into a thin fan or plane to reach and pass through the subject 28 and be detected by the detector 78.

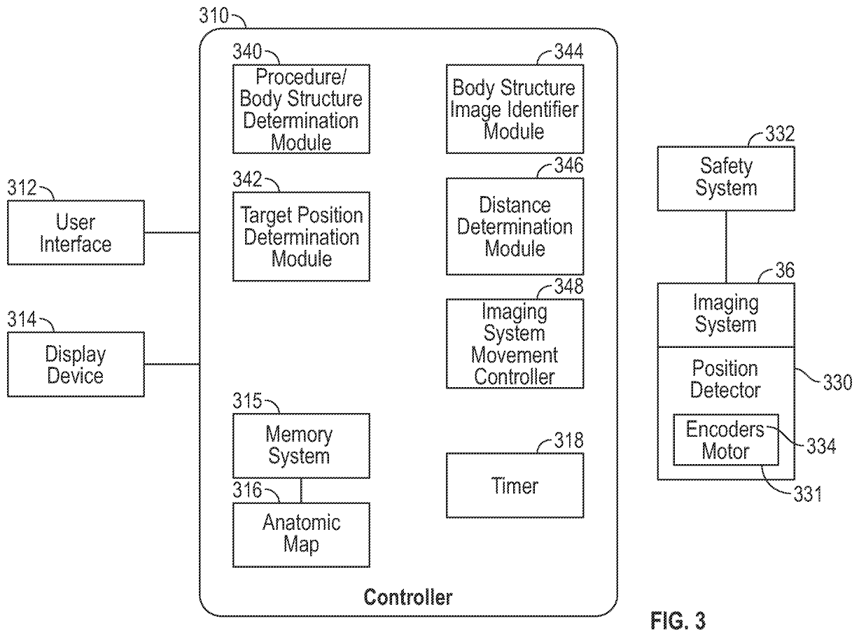

Referring now to FIG. 3, an example of a controller 310 that is programmed to execute instructions is set forth. The controller 310 may be one of or both of the processor 56 and the processor 68. The controller 310 is in communication with a user interface 312. The user interface 312 may be one of or both the user interfaces 52, 67. The controller 310 is also in communication with a display device 314 which may be one or both of the display devices 44, 69. In summary, the controller 310 may process various signals at the processor 56, the processor 68 or combinations thereof. Likewise, the user interface 312 may provide input to the controller 310 from the user interface 67 or 52. The display device 314 may display various features, images or data at the display device which may be 44 or 69 described above.

The controller 310 includes a memory system 315 that may be one of the memory system 66, the memory system 58 or a combination thereof. The memory system 315 is used to store various data including, but not limited to, the data described above relative to the memory system 66, 58. In addition, the memory system 315 may also be in communication with an anatomical map 316 stored therein.

A timer 318 is used to time various functions including the movement of the imaging system 316. That is, because a distance to the target and a characteristic of the movement for the system are known, the timer may be used to schedule the time desired to complete a particular movement. Further, the timer may be used to determine an length of time of a movement to determine when a selected position is reached given a selected velocity and/or acceleration of the system.

In the following example, the controller 310 is used to position the imaging system 36 which may be the O-arm® imaging system. Of course, other variations of the imaging system 36 may be used. The imaging system 36 may have a position detector 330 associated therewith. The position detector 330 is used for determining the relative position of the gantry 70 or movable structure of the imaging system. A position relative to a subject 28 of the imaging system 36, such as the detector 78, may be obtained. The position detector 330 may include one or more encoders 334 that are used to determine the amount of movement from a predetermined or an initial position. The encoder(s) 334 may include one or more encoders incorporated into a motor 331 that moves the imaging system, a counter (e.g., optical counter, magnetic) included relative to moving portions of the imaging system, etc.

A safety system 332 may also be associated with the imaging system 36. As mentioned above, the controller 310 may be used to automatically move the imaging system 36.

A safety system 332 may be associated with the imaging system 36. The safety system 332 may be one or more types of devices used for sensing the operating conditions of the imaging system 36. The safety system 332 may be a system having a collision sensor and/or a collision sensor used to sense the proximity to a person or equipment and generate a collision avoidance signal. Radar, lidar and ultrasonic sensors are examples. A no-fly zone may also be established around a patient and the equipment in the room. If the no-fly zone is entered by the imaging system, the imaging system may be programmed to stop moving. Likewise, a motor current sensor may be monitored on the movement motors, if a current threshold is exceeded, movement may be stopped, slowed or increase in a reverse direction. The safety system 332 may also be a mechanical switch such as a microswitch that is activated when touching something or a position accuracy sensor that determines whether the position from the position detectors 330 are providing accurate data. The safety system 332 may also include an override switch that allows an operator to stop the motion of the imaging system 36. In summary, the safety system 332 generates a safety system signal which, is monitored at the controller 310. In turn, the controller 310 may stop, slow or increase the speed of the movement of the imaging system when the signal is processed at the controller 310. The speed may include a linear and/or angular movement velocity of any selected portion of the imaging system.

The controller 310 has various modules that are used for allowing the imaging system 36 to be moved automatically. A procedure/body structure determination module 340 is used to determine or obtain a procedure or identify a body structure that is to be imaged. The procedure/body structure determination module 340 may include receiving input through a user interface 312. Selections or input signals from the user interface are used to select a procedure which, in turn, allows the target body structure that is to be imaged to be identified. A body structure may also be identified directly by a user through the user interface 312. For example, an initial image 40 or representation of a body structure may be displayed on the display device 314. A portion of the image or a representation of the image may be selected in a user interface.

By knowing the body structure to be imaged based upon the input at the procedure/body structure determination module 340, a target position determination module 342 is used to determine a target position for imaging the body structure of interest. Determining the target position takes into account various elements such as the position of the imaging system, the various angles that may be obtained by the imaging system and the beam generated by the imaging system, as set forth in FIG. 2 above. The beam may be a cone, fan beam, or a two-dimensional beam. The beam may be shaped and/or augmented with a selected collimator The target position is selected and/or determined as a position to position the imaging system 36 to acquire one or more image data projections of the subject to generate a selected image.

The imaging system 36 may be positioned into a position relative to the patient 28. The position may be detected by the position detector 330. Ultimately, the controller 310 determines the relative position to the patient 28 by a body structure imaging identifier module 344. The body structure image identifier module 344 receives an image from the imaging system 36 that is initiated by the user at the user interface 312. The imaging system 36 may provide an initial image or a series of initial images to the body structure image identifier module 344. The imaging system 36 may provide a two-dimensional image or a three-dimensional image or images to the body structure image identifier module 344. In some examples, a two-dimensional image may be provided to the body structure image identifier module 344. A two-dimensional image may have less data associated therewith and therefore allows a quicker processing time relative to a three-dimensional image. The amount of data depends in an image depends on but is not limited to the resolution, bit depth, number of projections and additional annotations/processing. The body structure image identifier module 344 identifies the one or more body structures that are present within the initial image using but not limited to machine learning image recognition, segmentation, comparison with an anatomic map. For example, features in the initial image may be segmented, using generally known segmentation techniques, and the segmented shapes or structures may be compared to the anatomic map to assist in identification of body structures in the image. The identified body structure in the initial image may or may not be the target body structure as discussed herein. Various body structures may be of interest depending upon the procedure to be performed. Likewise, in a post procedure situation, the body structure to be identified may be a body structure that a procedure has already been performed upon. For example, certain organs or bones may be identified. Likewise, markers may have been installed or implants may be implanted into a patient, such as a screw or the like. The bones may also include various vertebrae, an end plate, an edge of an end plate, a full vertebra, a partial vertebra, a skull, a limb, or an organ. Other examples include but are not limited to an end plate, an edge of an end plate, a full vertebra, a partial vertebra, a skull, a limb, or an organ.

A distance determination module 346 is used to determine a distance from an initial position to the target position. A distance determination 346 in conjunction with an imaging movement controller 348 positions the imaging system to move to the target position at a predetermined speed. The distance determination module 346 may determine a X, a Y and a Z distance that the imaging system 36 may need to move. Likewise and/or additionally, the distance may be a distance of movement of one or more various angles of the O-arm® imaging system around the axis 102 and the imaging system around the axis 106 may be used to position the cone of the collimator 220 in the target position and distances for movements of each of these may also be determined. It is understood that the distance that is determined may be a distance moved by any appropriate imaging system. Further, the distance that is determined may be a straight line movement distance and/or may be a non-linear movement distance that includes a non-linear path that includes more than one straight line movements and/or curve movements.

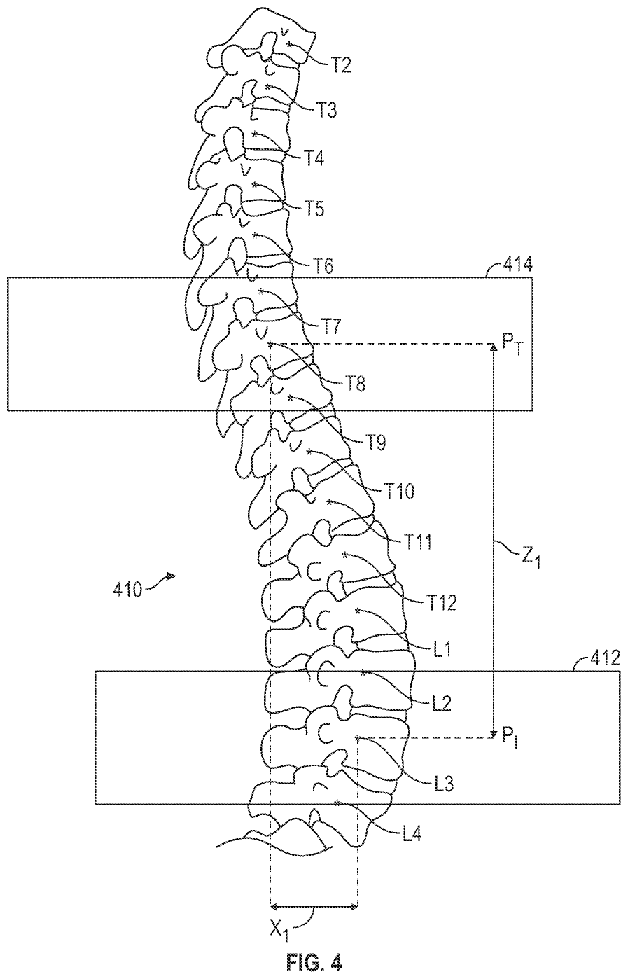

Referring now to FIG. 4, a body structure 410 is illustrated. The body structure 410, in this example, provides a plurality of thoracic vertebrae T2 through T12. Also, lumbar vertebrae L1-L4 are illustrated. In this example, the imaging system 36 may be located adjacent to the L3 vertebra at an initial position P_{I} and generates an initial image 412 represented by the box. In this example, the body structure image identifier module identifies the location and position of the vertebrate that the imaging system is directed toward. As mentioned above, this example includes the vertebrate L3 (e.g., being centered on L3). However, the procedure and body structure determination module 340 recognizes, such as through the user input, that the target image position PT is the desired position. The desired or target image position PT is vertebra T7. In this example, the imaging system 36 must move a distance Z₁. Also, the X direction X1 is illustrated between the lumbar L3 and thoracic T7 signifying the initial position P_{I} and target image position PT in the X direction. Of course, several images may be obtained such as anterior/posterior images (AP) or lateral images (LAT). Such images may also provide a movement direction in a Y direction. The target image 414 at the target position PT has an area represented by the box 414.

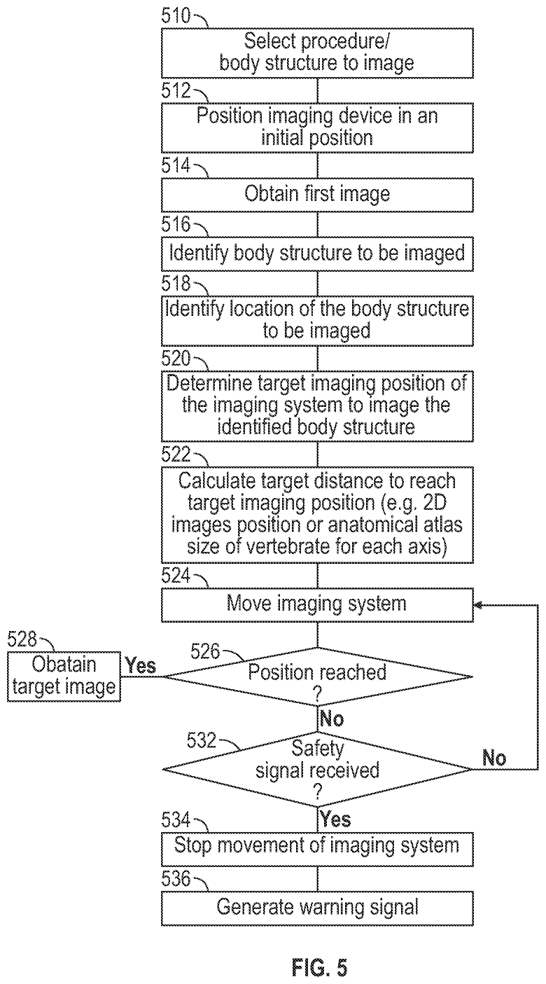

In other words, the imaging system may move in a plurality of movement directions and amounts to move the imaging system from a position to image the vertebra L3 to a position to image the vertebra T7. A method, as illustrated in FIG. 5, illustrates a method to move the imaging system to image a selected portion at the target position PT.

The images, according to various examples as discussed above, may be displayed on the display device 44, 69. Accordingly, the image data may be labeled image data for use by the user, such identifying portions in the image and/or portions of the geometry of the images portions. For example, the display device, which may be in the appropriate display device such as an LCD display, LED display, CRT display, or the like. Nevertheless, the image may be in the labeled image. Thus, the image may include labels of one or more vertebrae in the image data that is displayed as the image. The images or image may include the labels that are determined according to the various embodiments, as discussed herein.

The imaging system 36, or any appropriate imaging system, may be used to acquire image data of the subject 28. The image data may be analyzed, as discussed above, including labeling various features in the image data. The features may include anatomical portions in the image data, implants or surgical instruments in the image data or any other appropriate portion in the image data. According to various embodiments, various machine-learning systems, such as networks, may be trained to identify one or more features in the image data. As discussed above, the image data labels or identification may include centroids of vertebra. It is understood, however, that various portions of the image data may also be classified to be identified in the image data. Accordingly, during a selected procedure or at an appropriate time, image data may be acquired of the subject 28 with an appropriate imaging system, such as the imaging system 36, and features therein may be identified and/or labeled.

In various embodiments, a procedure may occur on the subject 28, such as placement of implants therein. Pre-acquired image data may be acquired of the subject, such as three-dimensional image data including a Computed Tomography (CT), Magnetic Resonance Imaging (MRI), or the like. The image data may be acquired prior to performing any portion of a procedure on the subject, such as for planning a procedure on the subject. The pre-acquired image data may be then used during a procedure to assist in performing the procedure such as navigating an instrument relative to the subject (e.g., a screw) and/or confirming a pre-planned procedure. In various embodiments, image data acquired of the subject during a procedure or after the acquisition of the initial or prior acquired image data may be registered to the prior or pre-acquired image data. For example, image data may be acquired with the imaging system 36 and may be registered to the pre-acquired image data according to various embodiments, as is generally understood by one skilled in the art. Registering a later acquired (e.g., a current image data or after procedure image data) may be registered to earlier acquired image data. The registration may allow a comparison of the earlier acquired image data (e.g., a plan) to a later acquired image data (e.g., post procedure) for determination of a and/or confirmation of a plan. The registration may be of one or more portions, such as one or more vertebrae portions.

Referring now to FIG. 5, a flowchart of a method for operating the system is set forth. In the following example, some of the blocks may be performed in various orders. In block 510, a selection of a procedure or body structure to image is generated. This may be an optional step. The generation of the structure to be imaged may be based on a selection signal, for example, which may be received from the user interface 312. The input from the user interface 312 may be of an identification of a procedure (e.g., selected vertebrae to be fused) which, in turn, will allow a body structure to be imaged to be identified.

In block 512, the imaging device is placed into an initial position. For example, the initial position may be saved or “zeroed out” relative to the positioning system 330, which may include the encoder. In various embodiments, the initial or start position may be saved for use in determining future positions of the imaging system 36 to achieve a selected position to acquire image data of the subject. In block 514, a first image is obtained at the initial position. As mentioned above, one or more two-dimensional images may be acquired or provided. The first image may be referred to as an initial image or a coarse image that may be in the vicinity but not at a target position. In block 516, the body structure of interest is identified based on input from the user. Machine learning or manual markers may be used to identify the particular body structure of interest. Machine learning output may have a definitive identification or several identification, one of which is identified by the operator. For example, a machine learning system may automatically output an identification based on selected inputs (e.g., training) and/or provide more than one output that may be selected by the user.

In block 518, a location of the target body structure is identified from the first image. The target body structure may be identified in coordinates relative to the initial position from measurements determined before imaging. For example, a X, Y and Z coordinate may be determined to obtain the desired target imaging position of the imaging device relative to the patient and/or a position of the imaging system 36. Likewise, the body structure itself may be identified by way of the user interface 312. For example, by touching of the body structure in the image 40 on the display device 44 with a finger, a stylus or a mouse pointer or touching the patient body with the instrument 144, the target body structure may be selected. In such a case, block 510 may be performed after the initial image is obtained. In block 520, the target imaging position for generating an image is determined. Calculations for the determination may be made based on distances such as those set forth in FIG. 4. For example, the distance in the Z axis may be used to determine a distance that the imaging system may need or move to be at the target position.

In block 522, a travel distance for an imaging system 36 to reach the desired imaging position is determined. Based upon the initial position of the imaging device in its initial or coarse position and the target imaging position for obtaining an image, a travel distance for the imaging system 36 to reach the desired imaging position is calculated and generated. The calculated travel distance may correspond to a distance in any or all of the axes. In other words, the travel distance may be a value of zero or more for each of the possible axes of movement of the imaging system. Likewise, rotational distance around the axis 106, or the user of rotation about the axis 102 may be taken into consideration.

The calculation of the travel distance may use an anatomical map, atlas data, or the size of the body structure such as the size of the vertebrae. The size of the body structure may be obtained from a previous three-dimensional image such as a CT or MRI image of the subject. For example, the CT image data may include spatial resolution in three dimensions of the patient. The CT image and the related size, therefore, may be recalled and/or analyzed for determining a current distance between points in a current image, especially when the two images are registered. The anatomical map may also provide the target imaging position for the image. In addition, a particular type of defect may be identified by the user interface without knowing the exact location. Global alignment parameters and measurements to determine a degree of coronal/sagittal deformity such as Cobb angles, lumbar lordosis, pelvic incidence, kyphosis, Central sacral vertical line (CSVL) are other examples of ways to identify a defect. For example, scoliosis or some type of defect may be sought. In this manner, the imaging system will evaluate the image data for certain parameters or features, such as a Cobb angle and/or CSVL, to identify certain body structures of interest having scoliosis or the selected defect until it is found using body recognition as described above. The target imaging position may therefore not be predetermined. A trained classifier, such as a neural network or machine learning may be used to identify a certain disease or body structure.

After block 522 determines the travel distance for the imaging system 36 to reach the target imaging position, the imaging system 36 is automatically moved in block 524. In block 524 the imaging system 36 is moved toward the target position with the ultimate goal of reaching the target position. The distance may include a linear distance in the X, Y and Z coordinates that may be moved depending on the movements achievable by a particular system to reach the target position. In addition, the distance may include an angular or rotational position of the gantry into a desired position may performed. The distance moved may include one or more of the distances. Including a combination of more than one such that the movement of the imaging system is not a single straight-line movement. In block 526, when the target position is reached, a target image or second image may be obtained from the imaging system 36 in step 528. The target image may be one or more of a two-dimensional or three-dimensional image. The target image is obtained and may be displayed for use by the user. For example, the target image may be used to confirm a procedure, diagnose a subject, and/or plan treatment for the subject.

In block 526 when the target position is not reached, block 532 determines whether a safety signal has been received. If a safety signal has not been received, the imaging system 36 continues to be moved toward the target position in block 524. In block 532 when a safety signal has been received, block 534 stops the movement of the imaging system. A warning signal in block 536 may be generated. The warning signal may be generated as an audible signal, a visual signal displayed on one of the displays or combinations thereof. A correction may be made or the processor system may be restarted after block 536. That is that the method may iterate to achieve the desired or target imaging position and/or may be stopped or restarted if a warning persists.

The system advantageously automatically moves the imaging system to the target location where an optimum target image may be obtained. The optimum target image is a second image obtained from a location that was desired to be viewed of the subject based on the input provided for the desired location. By providing rapid movement to a target position, exposure of the patient to X-rays may be minimized. Further, the amount of time to obtain the target image may be severely reduced.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors or processor modules, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A method of positioning an imaging system comprising:
positioning the imaging system;
acquiring a first image;
detecting a first body structure in the first image;
determining a first position of the imaging system relative to the first body structure based on the first image;
determining a distance to a target image position based on the relative position; and
moving the imaging system toward the target image position.

2. The method of claim 1 wherein acquiring the first image comprises acquiring the first image comprising a first two-dimensional image.

3. The method of claim 1 further comprising acquiring a second image at the target position.

4. The method of claim 1 further comprising segmenting the first image to identify the first body structure.

5. The method of claim 1 wherein determining the distance comprises determining the distance to the target image position based on an anatomical map.

6. The method of claim 1 determining the distance to the target image position includes determining a non-linear path.

7. The method of claim 1 further comprising determining a size of the first body structure based on a three-dimensional image and wherein determining the distance comprises determining the distance based on the size.

8. The method of claim 1 wherein determining the first body structure comprises determining at least one of an end plate, an edge of an end plate, a full vertebra, a partial vertebra, a skull, a limb, or an organ.

9. The method of claim 1 wherein determining the distance to the target image position based on the relative position comprises determining the distance to the target image position relative to a second body structure.

10. The method of claim 1 wherein moving the imaging system toward the target image position comprises moving the imaging system to the target image position.

11. The method of claim 1 wherein moving the imaging system toward the target image position comprises moving the imaging system while monitoring a safety system and generating a safety signal and changing the moving of the imaging system based on the safety signal.

12. The method of claim 1 wherein changing the moving of the imaging system comprises slowing the moving in response to a collision avoidance signal from a collision sensor.

13. The method of claim 1 wherein changing the moving of the imaging system comprises stopping the moving of the imaging system in response to a collision avoidance signal from a mechanical switch.

14. The method of claim 1 wherein changing the moving of the imaging system comprises slowing or increasing the moving of the imaging system in response to a collision avoidance signal from a mechanical switch.

15. The method of claim 1 wherein moving the imaging system comprises moving an O-arm of the imaging system.

16. A system to move an imaging system, the system comprising:
a controller configured to execute instructions to,
acquire a first image;
detect a first body structure in the first image;
determine a first position of the imaging system relative to the first body structure based on the first image;
determine a distance to a target image position based on the relative position; and
move the imaging system toward the target image position.

17. The system of claim 16 wherein the first image comprises a first two-dimensional image.

18. The system of claim 17 wherein the controller is configured to execute instructions to acquire a second image at the target image position based on the first image.

19. The system of claim 16 wherein the body structure comprises a vertebrate.

20. The system of claim 16 wherein the body structure comprises at least one of an end plate, an edge of an end plate, a full vertebra, a partial vertebra, a skull, a limb, or an organ.

21. The system of claim 20 wherein the controller is configured to execute instructions to determine the distance to the target image position based on the distance to the target image position relative to a second body structure.

22. The system of claim 21 wherein the controller determines the distance to the target image position based on an anatomical map.

23. The system of claim 16 wherein the controller determines a size of the first body structure based on a three-dimensional image and determines the distance based on the size.

24. The system of claim 16 wherein the controller is configured to execute instructions to move the imaging system to the target image position.

25. The system of claim 16 further comprising a safety system and generating a safety signal therefrom, the controller is configured to execute instructions to change the moving of the imaging system based on the safety signal.

26. The system of claim 25 wherein the safety system comprises a collision sensor.

27. The system of claim 25 wherein the safety system comprises a mechanical switch.
